(19) United States
(12) Patent Application Publication
(10) Pub. No.: US 2004/0229374 A1
(43) Pub. Date: Nov. 18, 2004

(54) System and method for determining fouling tendency by refinery feed stocks

(76) Inventors: Simon R. Kelemen, Annandale, NJ (US); Michael Siskin, Randolph, NJ (US)

Correspondence Address: EXXONMOBIL RESEARCH AND ENGINEERING COMPANY, P.O. BOX 900, 1545 ROUTE 22 EAST, ANNANDALE, NJ, 08801-0900, US

(21) Appl. No.: 10/721,958

(22) Filed: Nov. 25, 2003

Related U.S. Application Data
(63) Continuation of application No. 10/367,293, filed on Feb. 14, 2003, now abandoned.
(60) Provisional application No. 60/363,439, filed on Mar. 12, 2002.

(51) Int. Cl. G01N 33/00

(52) U.S. Cl. 436/139; 422/67

(57) ABSTRACT

The invention is a system and method to rate refinery feed stocks for the formation of deposits on solid refinery surfaces.

This is a Non-Provisional Application of Provisional U.S. Serial No. 60/363,439 filed Mar. 12, 2002.

### BACKGROUND OF THE INVENTION

The present invention relates to a system for rating refinery feed stocks, e.g., coker gas oils, catalytic cycle oils, atmospheric gas oils, coker naphthas, catalytic naphthas, steam cracked naphthas, feed stock mixtures and the like for the tendency to form deposits on solid refinery surfaces, e.g., in heat exchangers, inlet tubes, catalyst beds, etc.

The problem addressed by this system is deposits formed in refinery equipment. Such deposits cause operational problems.

Deposit mitigation by the use of additives is sometimes necessary, but testing the effects in the refinery equipment are laborious, time consuming and very expensive. Therefore, a rapid laboratory test, which ranks refinery feed stocks and refinery feed stocks containing additives in the order of deposit formation tendency and deposit mitigation effectiveness is of considerable value.

The present invention is a new way to make and study deposits from refinery feed stocks and additized refinery feed stocks. It is unique because its operating conditions can be changed to emulate the surface temperature fluctuations and feed transport behavior of the refinery equipment that the refinery feed stock and/or product will encounter.

### SUMMARY OF THE INVENTION

The present invention is a system and method to rate refinery feed stocks and refinery feed stocks containing additives for the tendency to form deposits. The system includes an optional enclosure, solid block (hereinafter called a “nub”) having a deposit surface within the enclosure, means for controlling magnitude and duration of the temperature of the deposit surface, means for introducing feed stock and/or feed stock containing additives, e.g. antioxidants used during transport or storage, into the enclosure onto the surface, and means for introducing gas into the enclosure if an enclosure is present. If the enclosure is omitted, then the deposits are, of course, in air. With an enclosure, the test may be performed in air or some other gas.

The nub is weighed before and after feed stock is placed onto the deposit surface. The change in nub weight indicates the feed stock and/or feed stock containing additives propensity to leave deposits.

### BRIEF DESCRIPTION OF THE DRAWINGS

FIGS. 1A and 1B show a schematic diagram of the system of the present invention.

FIG. 2 shows the variation in temperature of the deposit surface in time for Example 1.

### DETAILED DESCRIPTION OF THE PREFERRED EMBODIMENTS

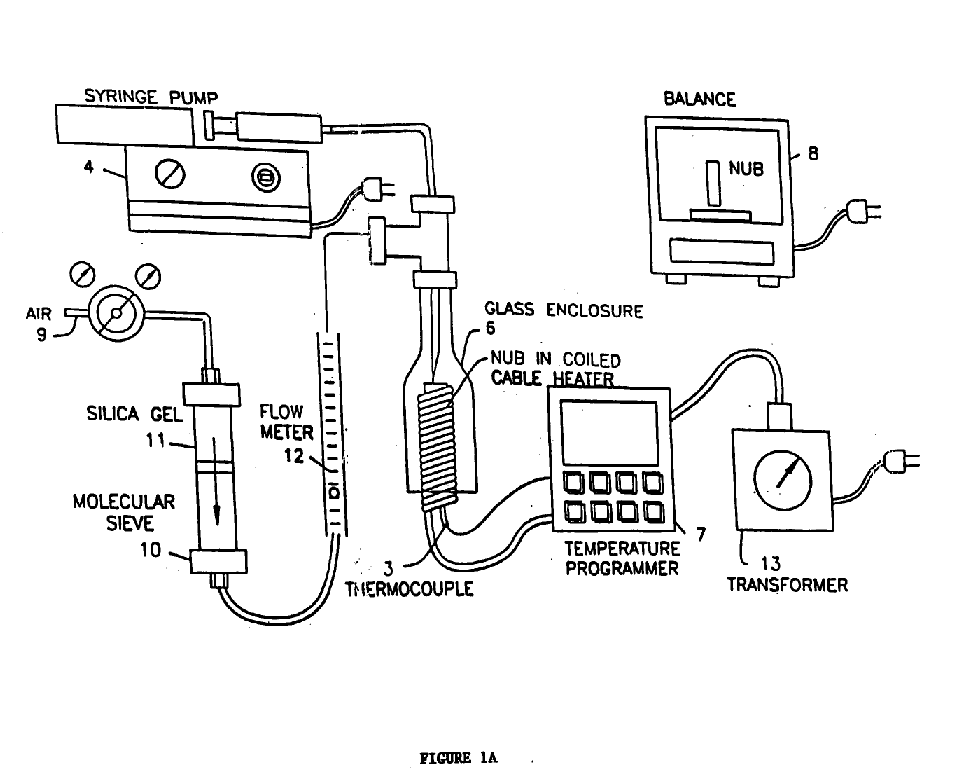

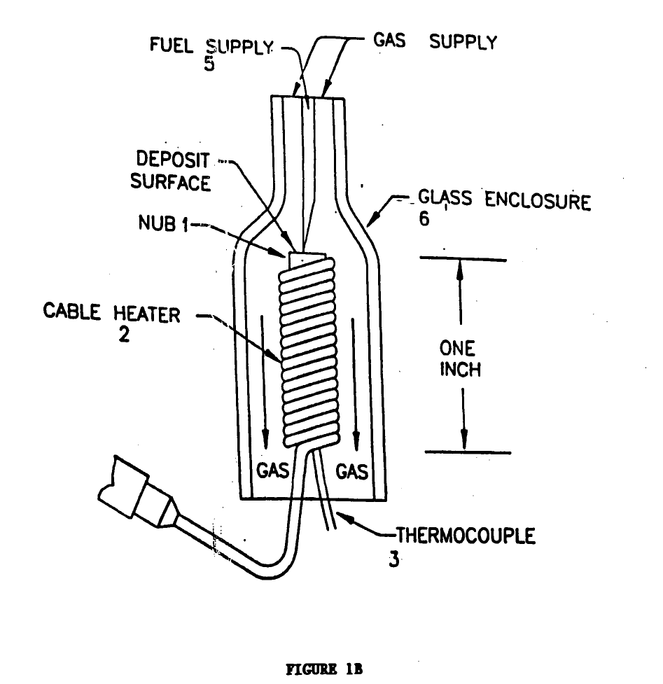

The system of the present invention is shown in FIGS. 1A and 1B. FIG. 1A shows a schematic view of the overall system. FIG. 1B shows an enlarged view of the nub [1] and thermocouple [3] arrangement. In the system, air or another gas [9] passes through a molecular sieve [10] that filters the air and removes contaminants. The air is dried by passing through silica gel [11].

The air is measured by the flow meter [12] and passes into the glass enclosure [6] where it combines with the feed stock that is to be rated. Within the glass enclosure [6], the system includes a nub [1] inside the coils of a cable heater [2]. The “nub” is formed of a solid material. A convenient shape is a solid circular cylinder. However, the shape, surface topography, and material of the nub deposit surface can be varied to simulate various surfaces of a feed stock system. Suitable materials include: steel, aluminum, and brass. A thermocouple is in close proximity with the nub-depositing surface, so as to control the nub surface temperature. A convenient way is to insert the thermocouple into a hole on the axis of the nub to a point under the deposit surface. The thermocouple [3] is used to control the deposit surface temperature. A novel feature of the present invention is that the deposit surface temperature is programmable [7]. With the aid of a transformer [13], the temperature can be steady or cycled through the range of temperatures encountered in various pieces of refinery equipment. The feed stock is delivered by a syringe pump [4] to the deposit surface through a hypodermic needle [5]. Like deposit surface temperature, the feed stock delivery rate can be programmed to emulate feed stock delivery rates to surfaces in various pieces of refinery equipment. A bell shaped glass enclosure [6] surrounds the nub and cable heater. It carries a blanketing flow of air [9], or any other desired gas, such as product gases, tail gases, recirculation gases, inert gases and the like to emulate refinery conditions and atmospheres. The system may be operated without the glass bell in which case the deposits occur in air. The nub is weighed by the balance [8] multiple times before and after each run to determine the average deposit mass accumulated onto the nub surface, which is typically 0.1 to 1.0 mg.

As shown in the Examples below, the system can emulate the deposit formation of feed stocks and products for refinery equipment. The operating conditions provide emulation of the deposit formation conditions for each piece of refinery equipment.

### EXAMPLE 1

The Present Invention Emulates Effects of Different Refinery Feed Stocks on Forming Foulant on a Metal Surface.

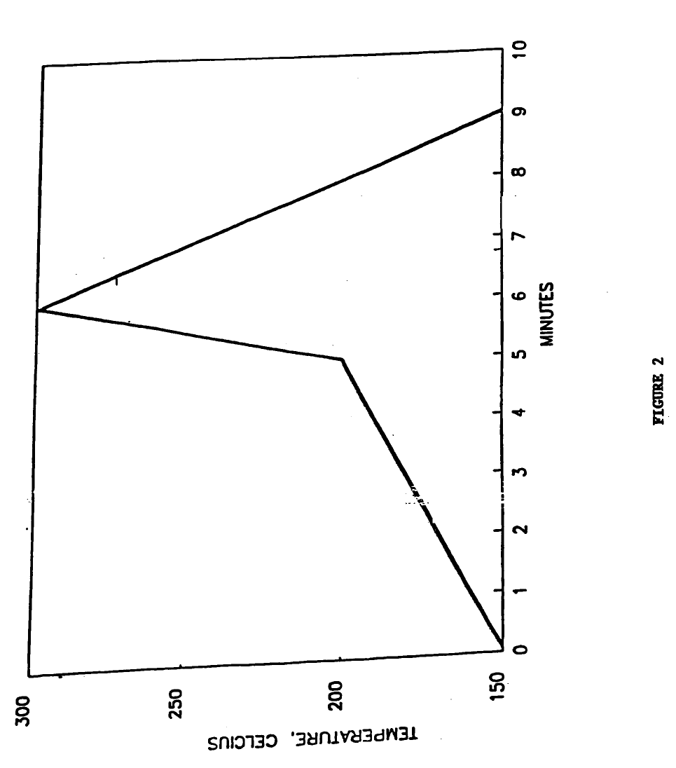

The procedure for making the deposit is as follows. A syringe pump (FIG. 1A) delivered test feed stock at a steady flow of 4 mL/hr for a test duration of one hour. During the one-hour test, the deposit surface temperature was programmed as shown in FIG. 2. This temperature cycle was varied between 150° C. and 300° C. The nub was weighed before and after the test. The difference in the nub weight is the total deposit weight, reported in units of milligrams per 4 mL of feed stock. The weight after washing the deposit with toluene is the toluene insoluble deposit weight reported below.

| | TOLUENE INSOLUBLE |
|---|---|
| SAMPLE | DEPOSIT (mg) |
| Refinery HCN T90 | 10.6 |
| Refinery HCN T90 | 9.7 |
| Refinery HCN T90 | 11.6 |
| Refinery HC01 | 3.1 |
| Refinery HC01 | 3.3 |
| Refinery LKGO | 5.7 |
| Refinery LKGO | 6.6 |

The results show that the Heavy Catalytic Naphtha (HCN) T90 Fraction of the total feed has the highest tendency to foul. It is significantly worse than the Light Coker Oil (LKGO) fraction of the total feed. However, when this HCN T90 fraction is blended in 10 wt % with other feed fractions in the HC01 total feed stock to the unit, its fouling tendency is much reduced.

### EXAMPLE 2

The Procedure of Example 1 was Repeated for 2 Hours at a Flow Rate of 4 ml/hr.

| | TOLUENE INSOLUBLE |
|---|---|
| SAMPLE | DEPOSIT (mg) |
| Final Mogas Product | 0.01 |
| Final Mogas Product | 0.00 |
| IBN HT 1st Stage Product | 0.29 |
| IBN HT 1st Stage Product | 0.18 |
| SCN (NO BHT) | 1.50 |
| SCN (NO BHT) | 1.28 |
| SCN (With BHT) | 1.10 |
| SCN (With BHT) | 1.25 |

A series of steam cracked naphthas were tested. The final hydrotreated naphtha product that goes into motor gasoline (Mogas) and shows almost no fouling tendency. The IBN HT product before final hydrotreating as a gasoline blending feed shows some fouling tendency, whereas the raw steam cracked naphtha (SCN), before any hydrotreatment has a high fouling potential; even when BHT is added as an antioxidant.

### EXAMPLE 3

The Procedure of Example 2 was Repeated on a Series of Steam Cracked Naphthas that were Hydrotreated to Remove Differing Levels of Styrene.

| | | TOTAL | HEPTANE | TOLUENE |
|---|---|---|---|---|
| | % STYRENE | DEPOSIT | INSOLUBLE | INSOLUBLE |
| SAMPLE | CONVERTED | (mg) | DEPOSIT (mg) | DEPOSIT (mg) |
| SCN | 100 | 0.46 | 0.15 | 0.14 |
| SCN | 88 | 0.88 | 0.62 | 0.52 |
| SCN | 65 | 1.29 | 1.08 | 0.78 |

The results show that the fouling tendency of the steam cracked naphtha is reduced as more styrene in the naphtha is hydrogenated. Also, washing the total deposit at room temperature with heptane removes some lower molecular weight material. Further, room temperature washing with toluene removes additional soluble material.

### EXAMPLE 4

The Procedure of Example 2 was Repeated on a Light Atmospheric Gas Oil (LAGO), a Blend of a Heavy Catalytic Naphtha With a Light Catalytic Cycle Oil (HCN/LCCO) and a Fresh Feed to a Diesel Hydrofiner. These Tests Were Run in an Air Atmosphere and Under a Nitrogen Atmosphere.

| | TEST | TOTAL | HEPTANE | TOLUENE |
|---|---|---|---|---|
| | ATMOS- | DEPOSIT | INSOLUBLE | INSOLUBLE |
| SAMPLE | PHERE | (mg) | DEPOSIT (mg) | DEPOSIT (mg) |
| LAGO | Air | 0.75 | 0.49 | 0.25 |
| LAGO | N₂ | — | 0.22 | 0.22 |
| HCN/LCCO | Air | 1.30 | 1.04 | 0.34 |
| HCN/LCCO | N₂ | 0.15 | 0.09 | 0.08 |
| Fresh Feed | Air | 5.83 | 0.62 | 0.60 |
| Fresh Feed | N₂ | 4.54 | 0.16 | 0.06 |

It is clear that maintaining a nitrogen atmosphere reduces the fouling tendency of the feeds and leads to lower levels of heptane and toluene insoluble deposits after room temperature washing. This example illustrates that the fouling tendency of feed stocks can be determined under varying atmospheric conditions and in this case running the test in air represents the most severe test; the maximum amount of foulant expected if the feed stock is not properly stored.

### EXAMPLE 5

The Procedure of Example 2 was Followed Oil a Set of Steam Cracked Naphthas from a Different Refinery.

| | TOTAL | HEPTANE | TOLUENE |
|---|---|---|---|
| | DEPOSIT | INSOLUBLE | INSOLUBLE |
| SAMPLE | (mg) | DEPOSIT (mg) | DEPOSIT (mg) |
| Final Mogas Product | 0.00 | 0.00 | 0.00 |
| 1st Stage Hydrotreated | 0.16 | 0.16 | 0.11 |
| SCN | | | |
| Raw SCN | 2.13 | 1.63 | 0.92 |

### EXAMPLE 6

The Procedure of Example 2 was Followed on a Commercial Premium Grade Motor Gasoline Containing all Required Additives.

| | TOTAL | HEPTANE | TOLUENE |
|---|---|---|---|
| | DEPOSIT | INSOLUBLE | INSOLUBLE |
| SAMPLE | (mg) | DEPOSIT (mg) | DEPOSIT (mg) |
| Premium Mogas - | 0.54 | 0.19 | 0.06 |
| Additized | | | |

The deposit formed in this case is essentially due to the additives in the motor gasoline. Most of the deposit is solubilized in heptane and toluene and would be expected to be more soluble at higher temperature.

1. A system to rate refinery feed stocks for the formation of deposits on solid refinery surfaces comprising:
(a) a solid nub having a deposit surface,
(b) means for controlling the magnitude and duration of the temperature of said surface, such that the temperature emulates the temperature variation of the solid refinery surface,
(c) means for introducing fuel and/or fuel containing additives onto said surface, and

2. The system of claim 1 further comprising a means for weighing said nub.

3. The system of claim 1 further comprising an enclosure and a means for introducing gas into said enclosure.

4. The system of claim 1 wherein said nub is steel, aluminum, brass or any solid material, or combination thereof.

5. The system of claim 1 wherein said means to control temperature includes a coiled cable heater, thermocouple and a temperature programmer.

6. The system of claim 1 wherein said means for introducing fuel and/or fuel additives, including a syringe pump and a hypodermic needle.

7. The system of claim 3 wherein said enclosure is a glass bell shaped shield.

8. The system of claim 3 wherein said gas is air.

9. The system of claim 3 wherein said gas is inert.

10. A method to determine the conditions for refinery feed stocks and/or refinery feed stocks containing additives for the formation and mitigation of deposits onto a solid in a controlled environment, having a surface of a given shape and material comprising:
(a) controlling the environment and magnitude and duration of the temperature of said surface such that the environment and the temperature emulates the environment and temperature variation of the solid refinery surface temperature of said surface,
(b) introducing refinery feed stocks and/or refinery feed stocks containing additives in controlled amounts onto said surface, and
(c) weighing said solid before and after said introducing step to determine the amount of deposit onto said surface.

11. The method of claim 10 further comprising the step of enclosing said solid.

12. The method of claim 11 further comprising the step of injecting a gas into said enclosure.

13. The method of claim 10 further comprising the step of programming the temperature of said surface.

14. The method of claim 12 wherein said gas is air.

15. The method of claim 12 wherein said gas is nitrogen.
